(19) United States
(12) Patent Application Publication
(10) Pub. No.: US 2023/0260632 A1
(43) Pub. Date: Aug. 17, 2023

(54) System and method for underwriting and financing of elective health procedures for clinical capacity optimization

(71) Applicant: Yes Doctor Corporation, Irvine, CA (US)

(72) Inventors: Amin Shameli, Irvine, CA (US); Behnam Analui, Irvine, CA (US); Hatem Abou-Sayed, Rancho Santa Fe, CA (US)

(73) Assignee: Yes Doctor Corporation, Irvine, CA (US)

(21) Appl. No.: 18/109,681

(22) Filed: Feb. 14, 2023

Related U.S. Application Data
(60) Provisional application No. 63/310,550, filed on Feb. 15, 2022.

(51) Int. Cl. G16H 40/20; G06Q 30/0235; G06Q 40/03

(52) U.S. Cl. CPC G16H 40/20; G06Q 30/0235; G06Q 40/03

(57) ABSTRACT

System and method for underwriting and financing of elective health procedures for clinical capacity optimization, including determining, for each of at least some available time slots in a calendar, an effective net profit as a function of a corresponding optimum discount rate, wherein the optimum discount rate for a calendar slot is the discount rate that maximizes an expected net profit of a clinic.

### COPYRIGHT STATEMENT

This patent document contains material subject to copyright protection. The copyright owner has no objection to the reproduction of this patent document or any related materials in the files of the United States Patent and Trademark Office but otherwise reserves all copyrights whatsoever.

### RELATED APPLICATION

This application claims the benefit of U.S. Provisional patent application No. 63/310,550, filed Feb. 15, 2022. Application No. 63/310,550 includes an Appendix which is part of that application. The entire contents of application No. 63/310,550, including the Appendix thereto, are hereby fully incorporated herein by reference for all purposes.

### FIELD OF THE INVENTION

This invention relates to the underwriting and financing elective health procedures for clinical capacity optimization.

### BACKGROUND

Many elective health procedures and emerging treatments are not covered by insurance, so patients have to self-pay for these procedures and treatments.

Many patients resort to financing options such as credit cards or personal loans to pay for such treatments. The cost of financing is high and is reflected in either a high-interest rate to the patient or a high provider fee in the form of a discount. In addition, financing typically includes a hard pull on the patient's credit report that affects the patient's credit score. The balance of the financed amount will also appear on the patient's credit report, further negatively impacting the patient's credit score.

In many cases, clinics offer potential patients financing options for procedures and treatments. Such financing options typically involve a lender, and, in most cases, the clinic needs to provide a discount to the lender to subsidize the interest rate paid by the patient. Therefore, the clinic incurs the cost of financing in addition to the marketing cost already incurred to attract the patient as a lead to the clinic.

Furthermore, in the elective health market, e.g., services provided by a plastic surgery clinic, the cost of service is typically very high for most of the population, and clinics have to offer frequent discounts to the patients to increase demand for their services to provide a continuous patient acquisition flow and to fill their capacity. However, the high cost of these services (e.g., plastic surgery services) usually requires significant discounts to be provided to patients, cutting significant profit margins from the clinics. A discount may need to be higher if the clinic needs to fill an immediate or near-future capacity that will otherwise remain unfilled, resulting in a lost revenue opportunity. Therefore, the clinics are constantly challenged to find the best discount to keep full capacity without sacrificing profit.

Another challenge for a clinic offering frequent discounts is the resulting market perception about the clinic's service prices and the consequent natural tendency of the market to continuously chase discounts with the clinic, which overall translates to lower average service prices.

In addition, given the clinics are responsible for selling the financing option, the cost of financing to the clinics disincentives the clinics from offering the financing to all their patients. As a result, an inherent adverse selection effect increases the cost of financing to the lender, which will propagate to the clinics, further discouraging the use and offering of such options.

Finally, even with significant discounts, most of the customer population may still be unable to pay out of pocket for clinic services; hence, the size of the potential customer pool for the clinics will remain limited.

In a two-sided marketplace with dynamic prices, an important problem that a service provider often faces is how to quote a price for a service to maximize profit. While a higher price will increase the revenue, a price that is too high may cause the client to abandon the transaction, and revenue is not realized at all. The problem is exacerbated and more complex in the markets where the service is not scalable, e.g., when a service provider has limited capacity or available time slots. In such scenarios, the service provider must also consider the probability of filling the capacity or time slot in the price quote. For example, in a plastic surgery clinic, the clinic's quote to a patient must consider the typical fees of the clinic for the treatment, the patient's capability to pay those fees, the time slot for the treatment, and whether the clinic can close the transaction with a different patient if the current patient is not closed.

The inefficiency and complexity of price and discount optimization may result in a loss of profit for clinics or unfair prices to the client.

Because they are not covered by insurance and are self-paid by patients, clinics that provide elective health procedures and emerging treatments leverage a standard open-loop sales funnel to acquire patients. Various fragmented marketing services generate patient leads for clinics. Clinics pay upfront for patient leads.

In today's market, clinics must rely on services from several disjointed third parties, such as marketing agencies, lead generation services, and financing firms, to acquire and serve patients. The lack of integration creates friction for patients, reduces conversion, and drives up patient acquisition costs for clinics. More recently, some newer technologies enable customizing or improving the potential patient lead experience. However, such technologies are only used for lead generation rather than complete patient acquisition, and they ignore some critical conversion steps. Clinics usually buy patient leads from various marketing services and pay upfront. However, patient leads may decide not to do a procedure. Worse, a patient lead may not even show up for a booked consultation appointment. The opportunity cost of a patient lead not appearing or not converting to a paying patient is high for the clinics but is typically not included in the total patient acquisition cost. The lead generation services are incentivized to produce as many leads as they can with limited to no consideration for the quality of the leads. They do not engage or impact the conversion of a lead to a paying patient.

The clinics must pay upfront for various fragmented marketing services for patient acquisition, and as a result, the patient acquisition cost is very high for clinics.

The clinic offers financing options. In most cases, the clinic needs to provide a discount to the lender to subsidize the interest rate paid by the patient. Therefore, the clinic incurs the cost of financing and the marketing cost already incurred to attract the patient as a lead to the clinic.

The combined effect has resulted in a small market share for financing and has brought predatory terms such as high-interest rates or deferred interest terms for patients.

Most elective health procedures and emerging treatments are offered by small or medium size clinics. The clinics that provide such services rely on open-loop patient acquisition funnels. The path of a potential patient through the funnel until committing and eventually completing treatment is typically called a patient journey. The clinic must continuously stay engaged with and support the patient throughout their journey since the cycle time of the journey may be long, and a potential patient may drop out of the funnel at any time for many reasons.

In today's market, the journey of a potential patient is typically managed through manual and/or disjointed steps by multiple people in the clinic or a mix of clinic and third-party service providers. For example, marketing agencies, lead generation services, customer relation managers (CRMs), and or practice management systems are separately utilized to manage the workflow for acquiring and providing service to patients. The engagement workflows are costly and inefficient for the clinic, are inconvenient, and provide a poor experience to the patient, overall driving the patient acquisition costs higher. The full-cycle workflow, during engagement with the lead and after converting to a patient and going through the treatments, requires the completion of multiple complex sets of interdependent, time-sensitive, and even unnecessary tasks at times. The inherent inefficiency in the system and managing the workflows further drive the costs higher for clinics and patients.

It is, therefore, desirable and an object hereof to provide a financing-driven discount optimization and capacity optimization method for clinics which results in the optimum discount offering, maximum capacity fulfillment, and overall higher profits realized for the clinics.

Other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification.

### SUMMARY

The present invention is specified in the claims and the description.

### BRIEF DESCRIPTION OF THE DRAWINGS

Various other objects, features, and attendant advantages of the present invention will become fully appreciated as the same becomes better understood when considered in conjunction with the accompanying drawings, provided by way of non-limiting examples, in which like reference characters designate the same or similar parts throughout the several views, and wherein:

FIG. 1 shows a framework according to exemplary embodiments hereof;

FIGS. 2A-2D show aspects of the platform of FIG. 1 according to exemplary embodiments hereof;

FIG. 3A shows aspects of an expected net profit optimizer mechanism according to exemplary embodiments hereof;

FIG. 3B depicts aspects of determining an optimum discount rate for available calendar slots according to exemplary embodiments hereof;

FIG. 4 shows aspects of the probability of filling a calendar spot;

FIG. 5 shows aspects of an underwriting engine according to exemplary embodiments hereof;

FIG. 6 shows aspects of a booking probability estimation model according to exemplary embodiments hereof;

FIG. 7 shows aspects of the probability of filing a calendar slot as a function of a discount rate;

FIG. 8 shows aspects of the effect on market demand of a discount provided to consumers and lenders;

FIGS. 9A-9C show aspects of booking flows according to exemplary embodiments hereof;

FIGS. 10A-10E show aspects of two-sided market negotiation and optimization according to exemplary embodiments hereof;

FIGS. 11A-11F depict aspects of integrated patient acquisition with closed-loop global optimization according to exemplary embodiments hereof;

FIGS. 12A-12B depict aspects of financing-driven marketing and financing prequalification for patient acquisition according to exemplary embodiments hereof;

FIGS. 13A-13C aspects of automating and optimizing the full-cycle workflow of engaging, acquiring, and serving patients according to exemplary embodiments hereof; and

FIG. 14 depicts aspects of a computer system according to exemplary embodiments hereof.

### DETAILED DESCRIPTION OF THE PRESENTLY PREFERRED EXEMPLARY EMBODIMENTS

In the following, exemplary embodiments of the invention will be described, referring to the figures. These examples are given to provide a further understanding of the invention without limiting its scope.

A series of features and acts are described in the following description. The skilled person will appreciate that unless the context requires explicitly, the order of features and steps is not critical for the resulting configuration and its effect. Further, it will be apparent to the skilled person that irrespective of the order of features and acts, the presence or absence of time delay between acts can be present between some or all of the described acts.

Reference numerals have just been referred to for quicker understanding and are not intended to limit the scope of the present invention in any manner.

### Glossary & Abbreviations

As used herein, unless used otherwise, the following terms or abbreviations have the following meanings:

A “mechanism” refers to any device(s), process(es), routine(s), service(s), or a combination thereof. A mechanism may be implemented in hardware, software, firmware, using a special-purpose device, or any combination thereof. A mechanism may be integrated into a single device, or it may be distributed over multiple devices. The various components of a mechanism may be co-located or distributed. The mechanism may be formed from other mechanisms. In general, as used herein, the term “mechanism” may thus be considered to be shorthand for the term device(s) and/or process(es) and/or service(s).

### Description

The following detailed description is not intended to limit the current invention. Alternate embodiments and variations of the subject matter described herein will be apparent to those skilled in the art.

Exemplary methods and systems according to exemplary embodiments hereof are described with reference to the drawings.

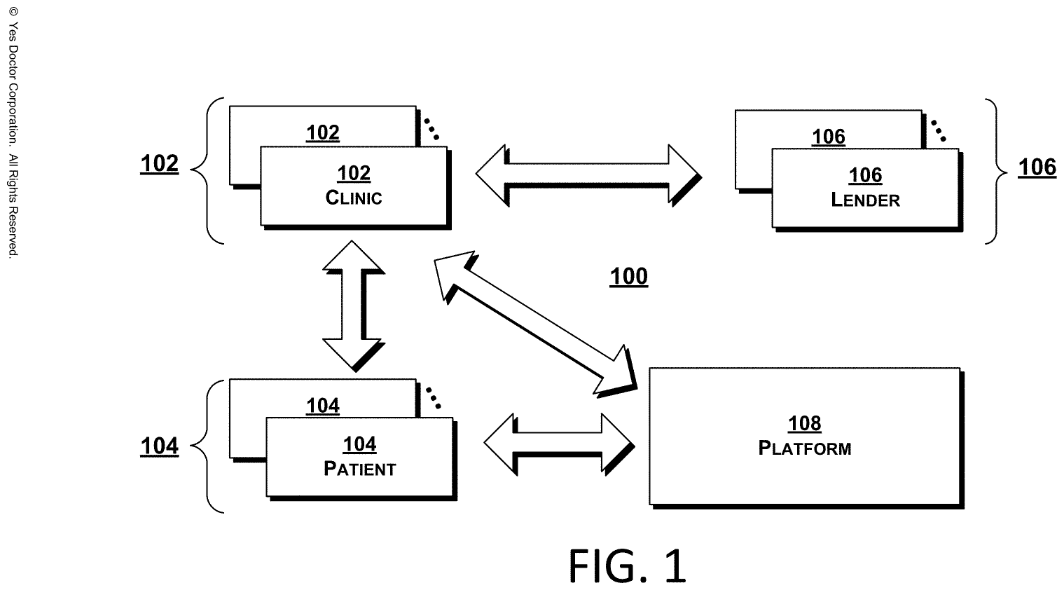

With reference to the drawing in FIG. 1, in a system 100, one or more clinics 102 provide services (e.g., elective health procedures and emerging treatments) to patients 104. The clinic(s) 102 may provide financing options (e.g., loans) to patients 104. While the loans may be provided by the clinics 102, the financing associated with the loans may be supported by one or more lenders 106.

As used herein, the term “clinic” generally refers to any entity that provides services or procedures and is not limited to any particular legal structure or entity. The organizational structure of any clinic does not limit the scope of the invention.

As used herein, the “services” provided by a clinic may include medical or health procedures, e.g., elective health procedures and emerging treatments. It should be appreciated that these are just examples, and the nature or type of procedures or treatments does not limit the scope of the invention.

As used herein, the term “patient” refers to a person that desires or intends to have one or more services or procedures. Since patients may not yet have had a procedure or service, they may be considered and referred to as potential patients.

As used herein, the term “lender” generally refers to any entity that lends money or supports the lending of money. A “lender” is not limited to any particular legal organization (e.g., a bank) or any particular kind of entity. The scope of the invention is not limited by the organizational or legal structure of any lenders.

A clinic 102 may provide a discount to a lender 106 to subsidize an interest rate paid by a patient 104. For example, a particular clinic 102 may lend a particular patient 104 an amount of $10,000 at X percent interest for a certain procedure. To provide the loan to that particular patient at that interest rate (X %), the clinic 102 may have engaged with a lender 106 and offered that lender 106 a discount (e.g., an amount such as, e.g., $1,000 or a percentage such as, e.g., 10%) for the loan. In such an arrangement, the patient 106 borrows from the clinic 102 (and therefore owes the clinic the borrowed amount, $10,000, plus interest), but the lender 106 gives the clinic 102 the discounted loan amount (e.g., $10,000 less the $1,000 discount). In some cases, the lender 106 may immediately “buy” the loan from the clinic 102 at the discounted rate.

A clinic 102 may have one or more practitioners (e.g., medical doctors, technicians, etc.), and these practitioners may offer various services or procedures (e.g., surgery, medical treatments, etc.). In a particular clinic, not all practitioners offer all services. For example, a plastic surgeon may not perform laser hair removal, and a technician should not perform plastic surgery.

A platform 108 (described in greater detail below) interfaces with clinics 102 and patients 104.

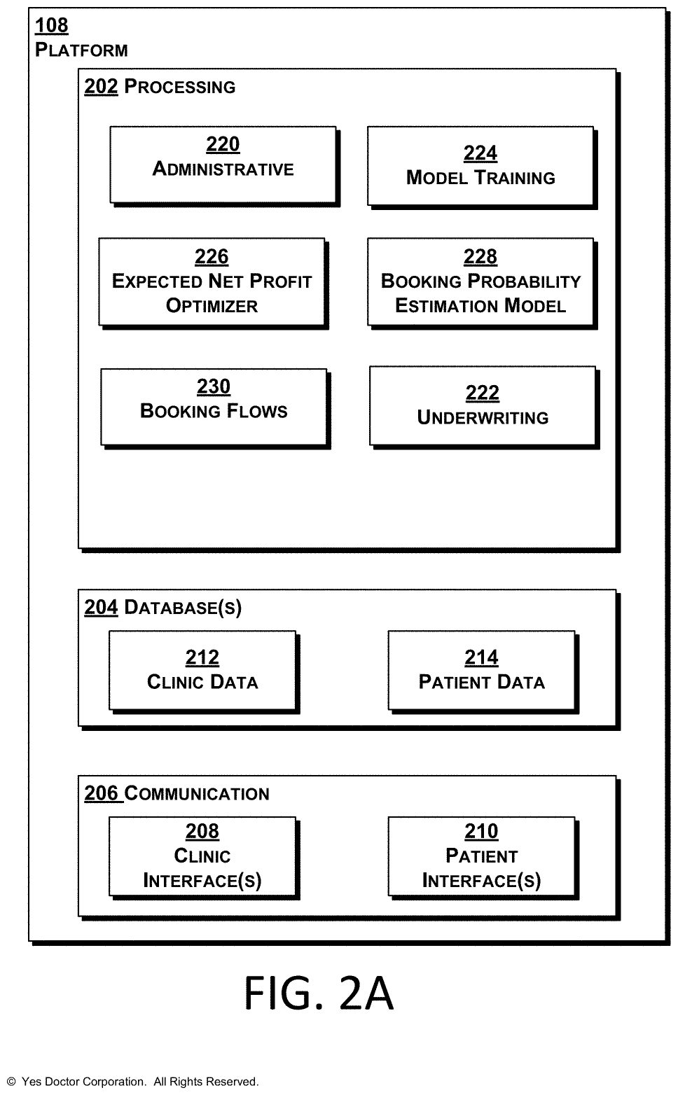

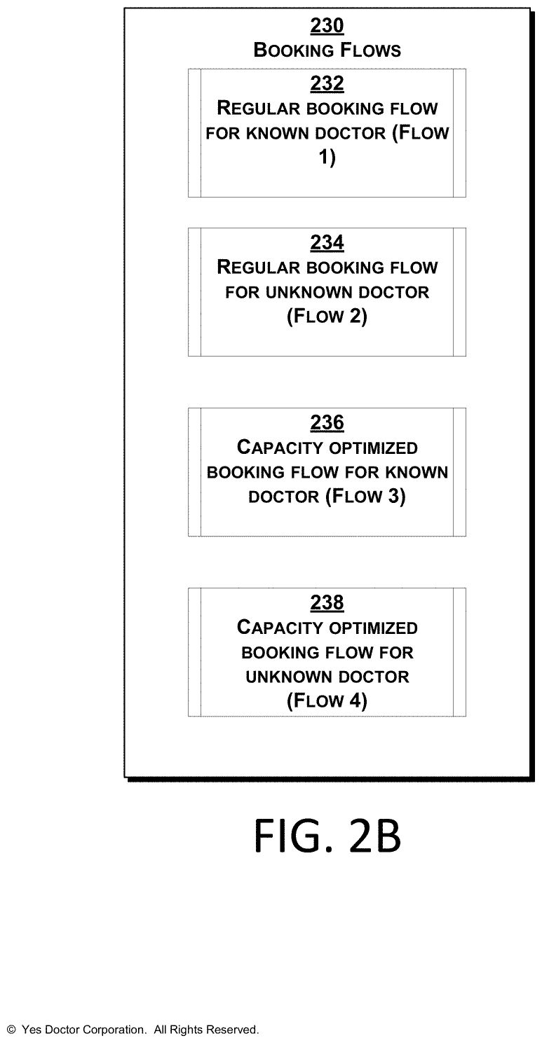

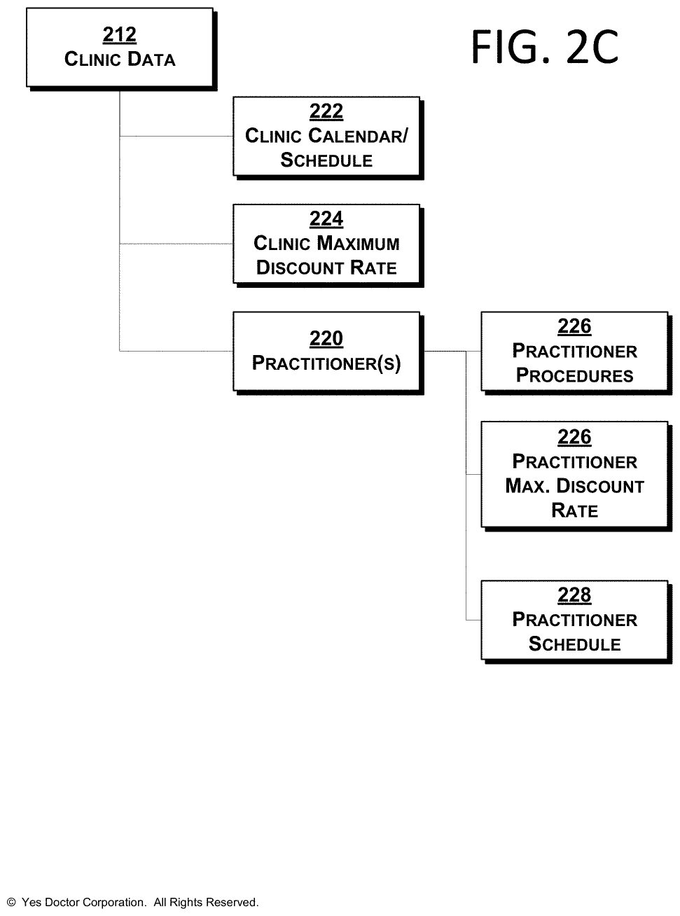

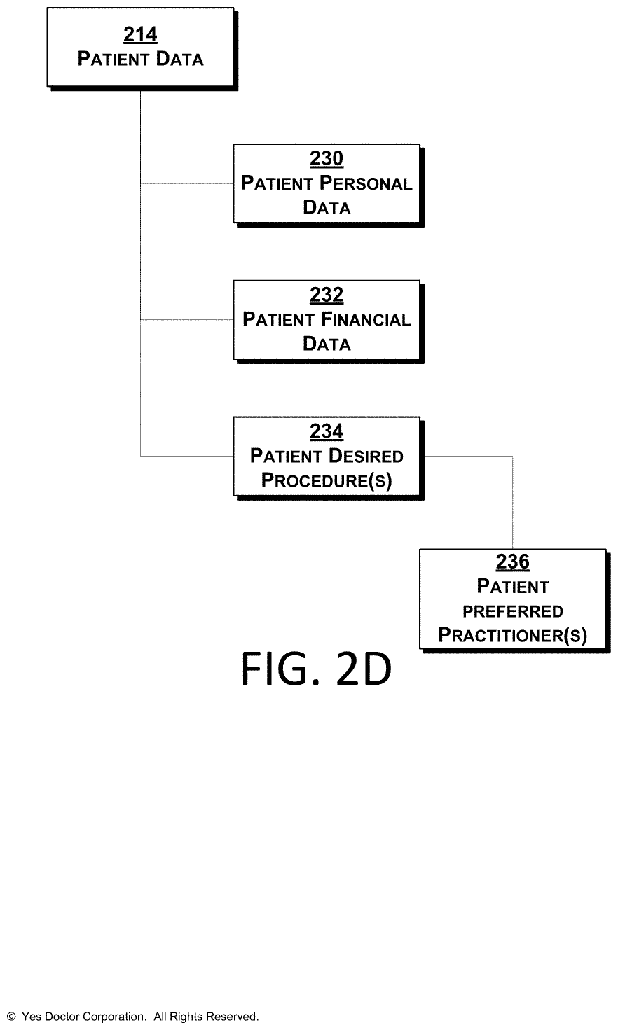

With reference to FIG. 2A, an exemplary platform 108 preferably includes processing mechanisms 202, one or more databases 204, communication mechanism(s) 206, and other miscellaneous mechanisms (not shown).

The processing mechanisms 202 may include mechanisms that provide and/or support the functionality described below, including at least the following functionality: administrative mechanism(s) 220, underwriting mechanism(s) 222, model training mechanism(s) 224, expected net profit optimizer mechanism(s) 226, booking probability estimation model mechanism(s) 228, and booking flow mechanism(s) 230.

With reference to FIG. 2B, the booking flow mechanism(s) 230 may include to following mechanism(s): regular booking flow for a known doctor 232, a regular booking flow for an unknown doctor 234, a capacity-optimized booking flow for a known doctor 236, and a capacity-optimized booking flow for an unknown doctor 238.

The processing mechanisms 202 may be implemented in hardware, software, firmware, or combinations thereof. The various listed processing mechanisms 202 and their logical organization is only exemplary, and different and/or other processing mechanisms may be included, having different and/or other logical organizations.

The various processing mechanisms 306 may include associated data stored in one or more data structures in the database(s) 204. The various data and their logical organization described here are only exemplary, and different and/or other data may be included, having different and/or other logical organizations.

The communication mechanism(s) 206 preferably includes mechanism(s) supporting clinic and patient interactions and interfaces 208, 210. Clinics 102 will typically have different interfaces and interactions than patients.

In a system 100, each clinic 102 preferably registers with the platform 108. It provides the platform with information about the services provided by that clinic, preferably at the granularity of which practitioners provide which services. Clinics may provide information to the platform 108 via the clinic interface(s) 208. For example, a particular clinic 102 may have ten practitioners (P1, P2 . . . P10), and, for each of the practitioners, the clinic may inform the platform 108 of what services that practitioner performs. For scheduling purposes, the clinic may also inform the platform of the expected amount of time for each service and the cost of that service.

Each clinic (or participating entity or practitioner) provides the platform 108 with a maximum discount rate, e.g., as a percentage, that they are willing to offer to the lender(s), preferably per participating doctor or clinic. For example, a clinic may set a maximum discount rate for the entire clinic (to cover all practitioners in the clinic), or it may provide a maximum discount rate per participating practitioner in the clinic. In this manner, different practitioners (e.g., doctors) can set their own maximum discount rates. As should be appreciated, the maximum discount rate covers the discount that the clinic (or practitioners) are given to the lender(s).

Each clinic 102 provides the platform 108 with a regularly updated schedule (e.g., calendar) showing available time slots by procedure and/or practitioner. For example, a particular clinic 102 may provide a schedule that shows that certain practitioners have available time slots on certain days (preferably including today and the next few days) for certain procedures. Or the particular clinic 102 may provide a schedule that shows the availability for certain procedures in the next few days without specifying which practitioners are available.

Each clinic's data may be stored in the clinic data 212 in the database(s) 204. With reference to FIG. 2C, exemplary clinic data 212 may include a clinic calendar or schedule 222, a maximum discount rate 224, and a list of one or more practitioners 220. The clinic data 212 may include other data (e.g., administrative data) not shown. For each practitioner, the clinic data 212 may include a list of the procedures that the practitioner performs 226, a maximum discount rate that the practitioner is willing to accept 226, and the practitioner's schedule 228. The practitioner's maximum discount rate 226 is preferably not more than the clinic's maximum discount rate 224. The practitioner's schedule 228 may be determined from the clinic's schedule 222. The clinic's schedule may also include time slots for procedures not associated with any particular practitioner.

Patients 104 (or potential patients) may register with the platform 108 via one or more patient interfaces 210. Patients 104 provide sufficient information to the system to enable or allow them to be matched with services (e.g., treatments or procedures) offered by the clinics 102.

Each patient's data may be stored in the patient data 214 in the database(s) 204. With reference to FIG. 2D, exemplary patient data 214 may include patient personal data 230, patient financial data 232, a list 234 of one or more procedures the patient desires, and a list of one or more preferred practitioners 236. The patient data 214 may include other data (e.g., administrative data) not shown.

For example, a patient may inform the system that they want a particular medical procedure, and that information will be stored in a record associated with that patient.

In operation, the platform 108 may try to match patients 104 with clinic's (or practitioner's) available (unfilled) schedule slots.

For example, a clinic 102 may provide the platform 108 with a schedule of open time slots for particular procedures. This schedule may be provided for the entire clinic or individually for practitioners in the clinic. The schedules are updated regularly, preferably at least daily. The schedules may be updated in real-time to reflect changes. An open time slot represents the availability of the clinic and/or a particular practitioner at the clinic to perform a certain procedure or treatment. For example, an open time slot may represent the availability of a particular physician to perform a particular plastic surgery procedure.

A clinic desires to fill each open slot profitably. To this end, as described here, the platform 108 may determine effective net profit for each calendar slot as a function of a discount rate (to be offered by the clinic to the lender). In this manner, patients may be matched with slots in a manner that provides an optimized discount rate per slot.

Unless otherwise noted, the discount and discount rate in the platform and as discussed below refer to the discount of the clinic to the lender.

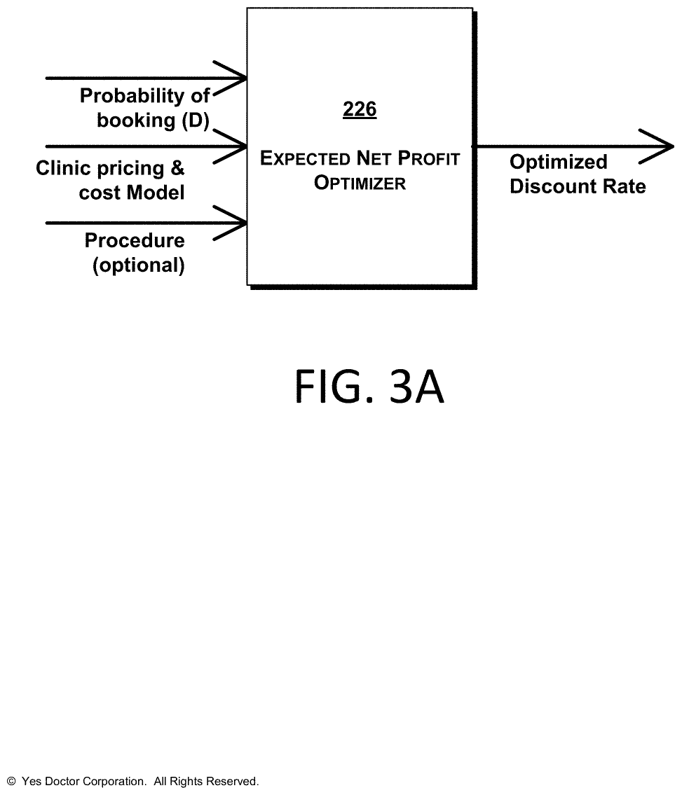

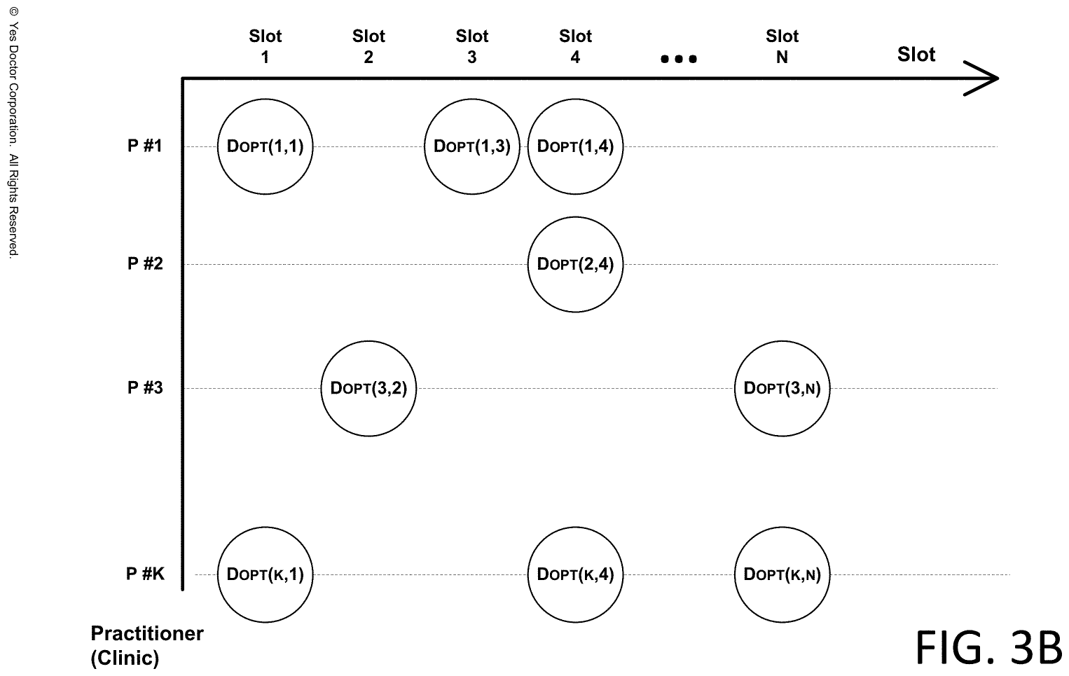

With reference to FIGS. 3A-3B, the expected net profit optimizer mechanism(s) 226 may identify or calculate the optimum discount rate for a given calendar slot and, given its probability of booking, by optimizing the expected net profit of the clinic.

As shown in FIG. 3B, an optimum discount rate Dₒₚₜ(x, y) is determined for each available calendar slot (for the practitioner (or clinic) x, time y). Thus, e.g., practitioner P #3 has time slots at time (slot) #2 and #N. The expected net profit optimizer mechanism(s) 226 determines an optimum discount rate for each of those slots (namely, Dₒₚₜ(3,2) and Dₒₚₜ(3, N)).

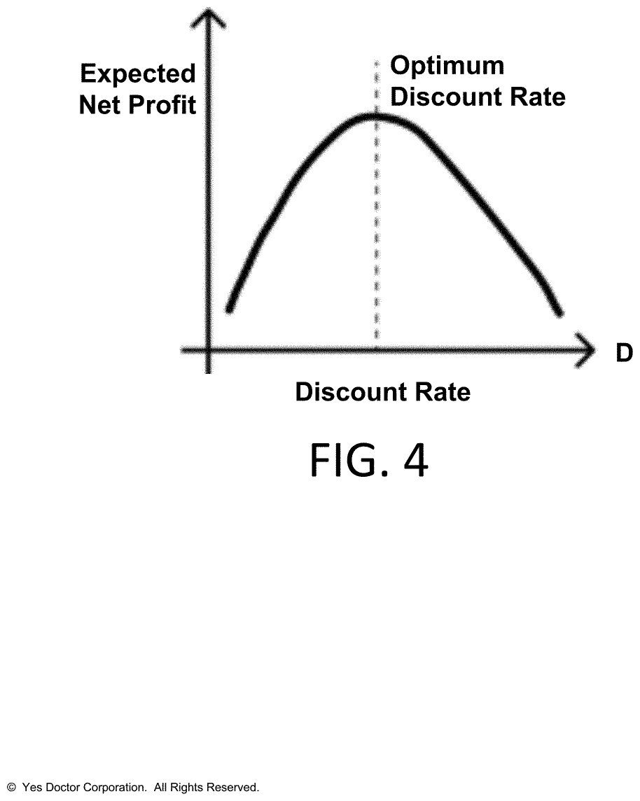

The net profit of a clinic may be a function of the discount rate that can be calculated from two functions: (i) the profit of the clinic conditioned or given the calendar slot is booked, and (ii) the probability of booking the calendar slot, where (i) is a decreasing function of the discount rate and (ii) is a monotonic and non-decreasing function of the discount rate. Therefore, the expected net profit of a clinic for a given calendar spot as a function of the discount rate is expected to have a maximum, e.g., as shown in FIG. 4 (showing the expected net profit for a given calendar spot as a function of the discount rate). The optimum discount rate is the discount rate that results in the maxima.

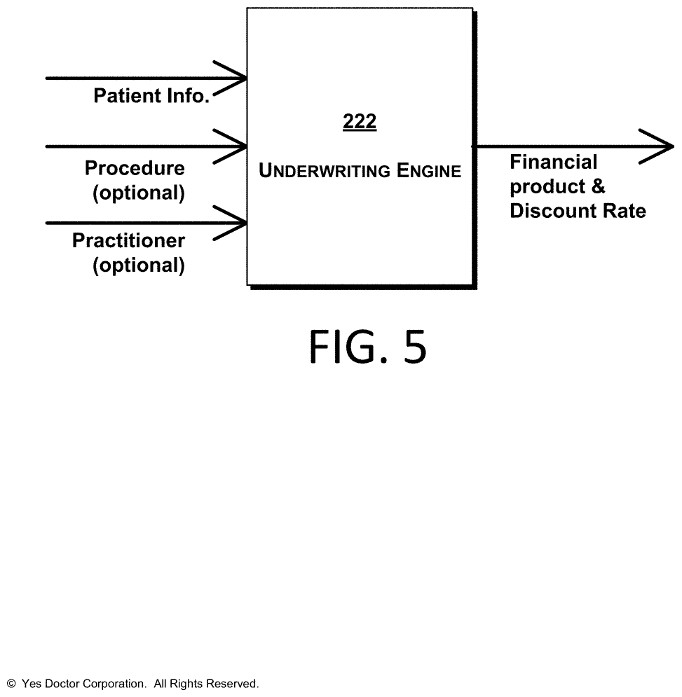

For a given patient, the underwriting engine (or mechanism(s)) 222 (FIG. 5) may identify or calculate (or determine) the possible financial products and a minimum required discount rate needed for a potential patient.

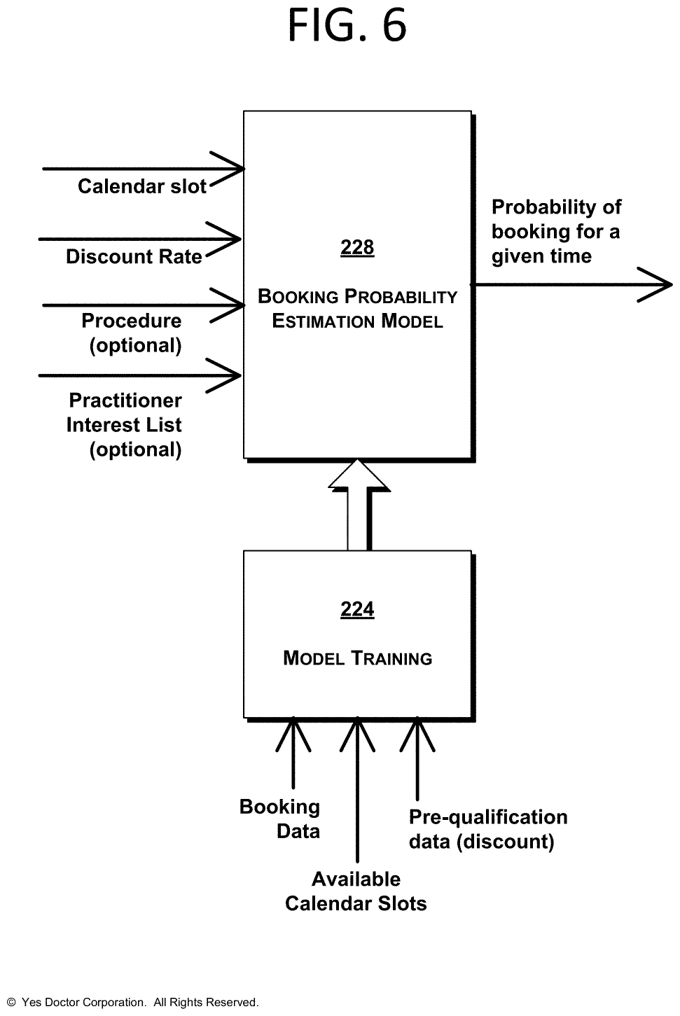

The platform's booking probability estimation model mechanism(s) 228 (FIG. 6) may identify or calculate the probability of booking for a given unfilled capacity slot of a given clinic as a function of the discount rate that the clinic (or practitioner) offers.

The booking probability estimation model 228 may be determined using model training mechanism(s) 224. The probability of booking for a given time slot may be estimated based on a machine learning or other algorithm or from past data of the clinics or may be learned over time.

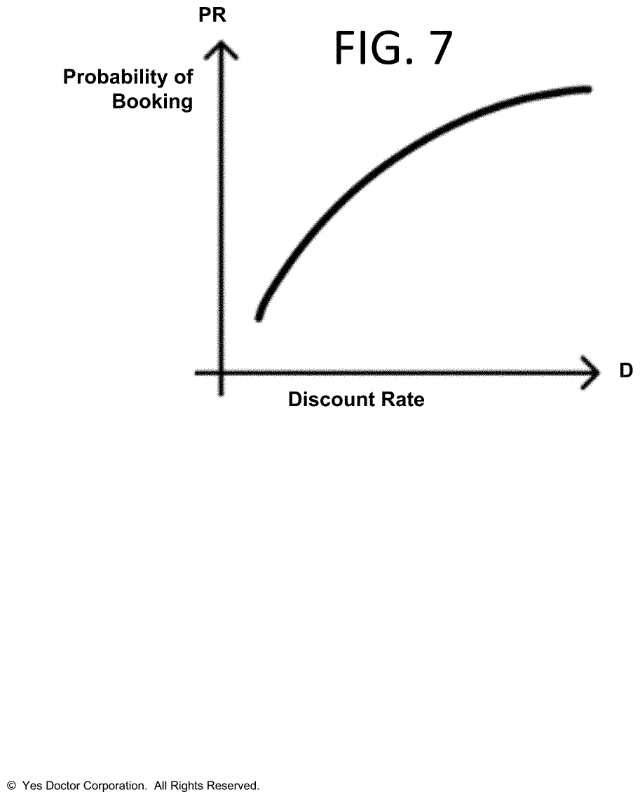

The probability of booking or filling a calendar spot is typically a monotonically non-decreasing function of the discount rate, e.g., as shown in FIG. 7.

The platform 108 may support multiple booking flows to serve various patients, depending on the patient's minimum required discount rate, optimum discount rates, and or maximum discount rates that practitioners or clinics are willing to offer.

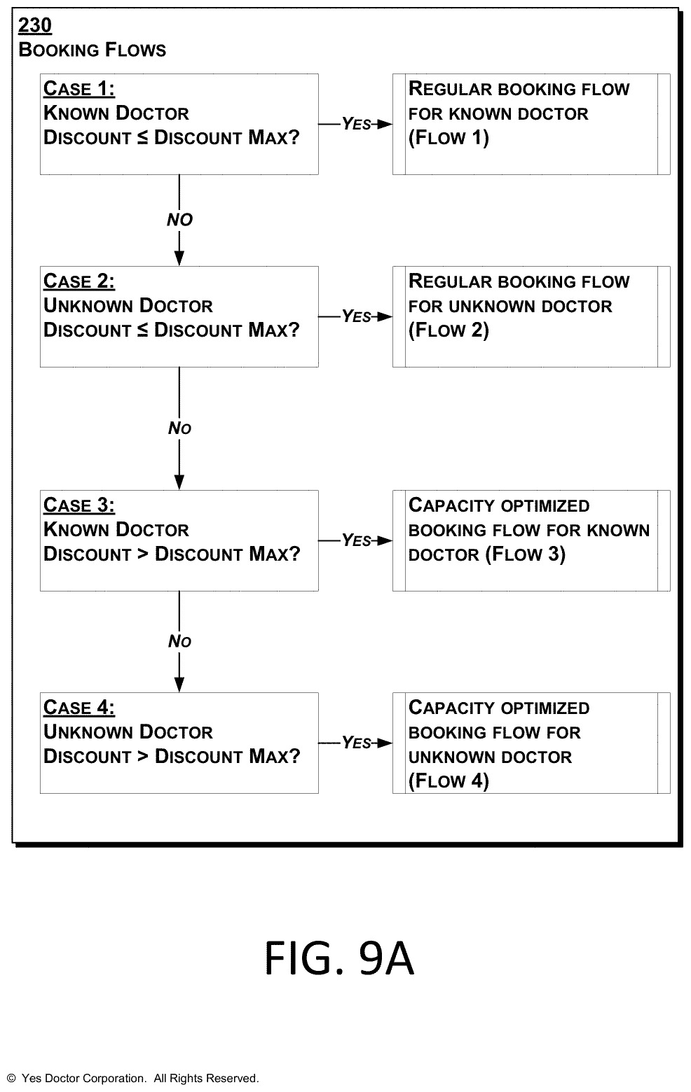

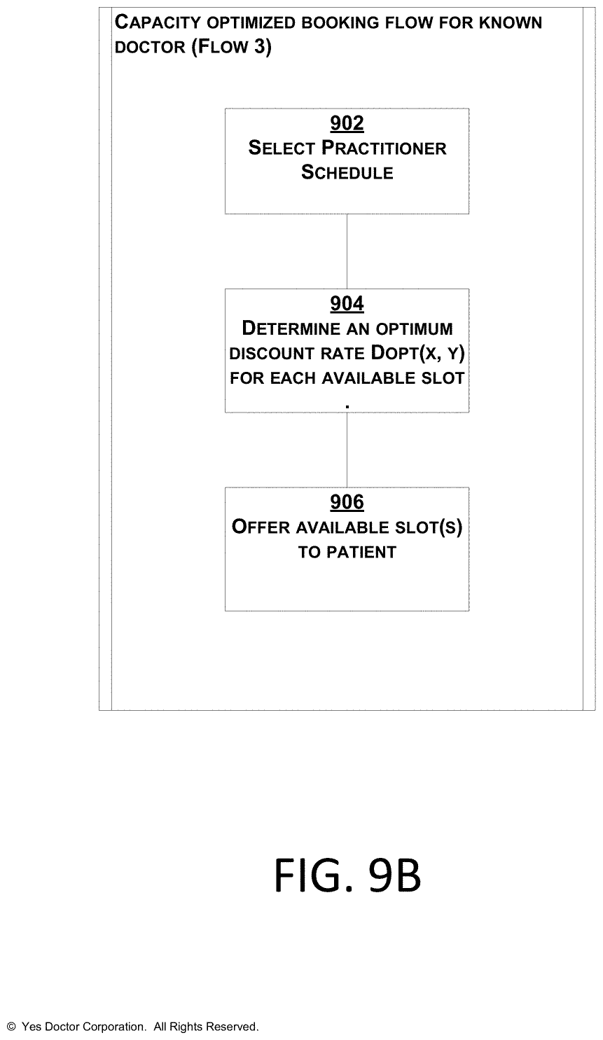

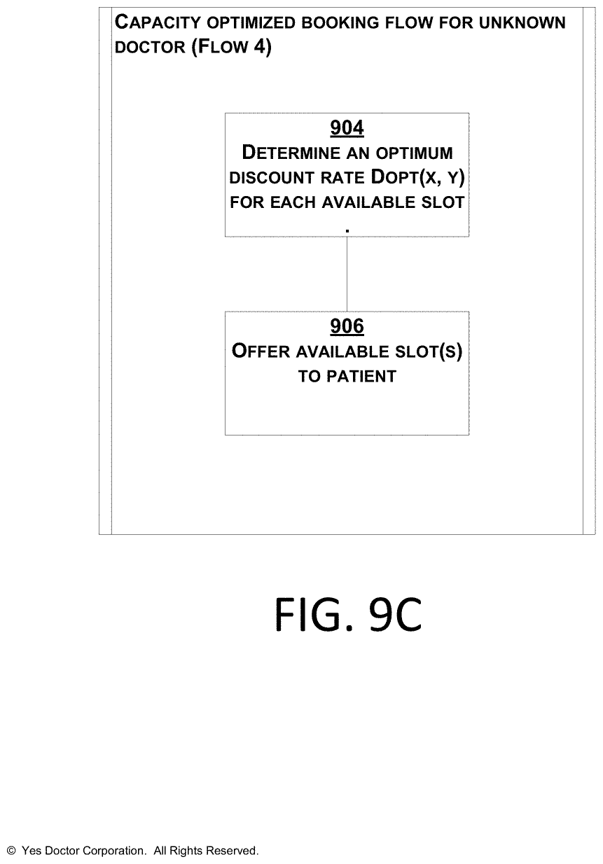

Various booking flow mechanism(s) 230 are shown in FIG. 2B, corresponding, e.g., to the booking flows 230 shown in FIG. 9A. In the booking flow shown in FIG. 9A, the patient wants a specific doctor (practitioner), in which case the flow refers to a “known doctor.” In cases #1 and #3, the patient wants a specific or “known” doctor.

If the patient has good credit, they are likely to get a loan at a good rate and may not even require a discount. However, if the patient has poor credit (e.g., a low credit score), the lender may require a greater discount from the clinic to support or provide the loan. Recall that the clinic gives the loan to the patient, but the clinic gets the loan (or a discounted loan) from the lender by providing a discount to the lender. The lower the patient's creditworthiness, the higher the discount will be. In cases #3 and #4, the platform 108 performs capacity-optimized booking (either for a known doctor—flow 3 or for an unknown doctor—flow 4). An outcome of these two flows is the determination of an optimal discount rate for that patient.

With reference to FIGS. 3B and 9B, in case #3 (“known doctor”), the patient has a preferred one or more practitioners, so the optimal discount rate may be determined for just those practitioners. As shown in the flow chart in FIG. 9B, first one or more practitioner schedules are selected (at 902). These schedules may correspond to the rows in FIG. 3B and are selected for the patient's preferred or required practitioners. Then an optimum discount rate Dopt(x,y) is determined for each available slot for those practitioners (at 904). If one or more slots are available for the patient at an acceptable discount rate for the clinic/practitioner, then the patient is offered those slots (at 906).

In case #4 (“unknown doctor”), the patient has no preferred practitioner, so the optimal discount rate may be determined for all open slots that might work for that patient. With reference to FIGS. 3B and 9C, the processing is similar to case #3 (FIG. 9B), except that the optimum discount rate is determined for all slots for all practitioners.

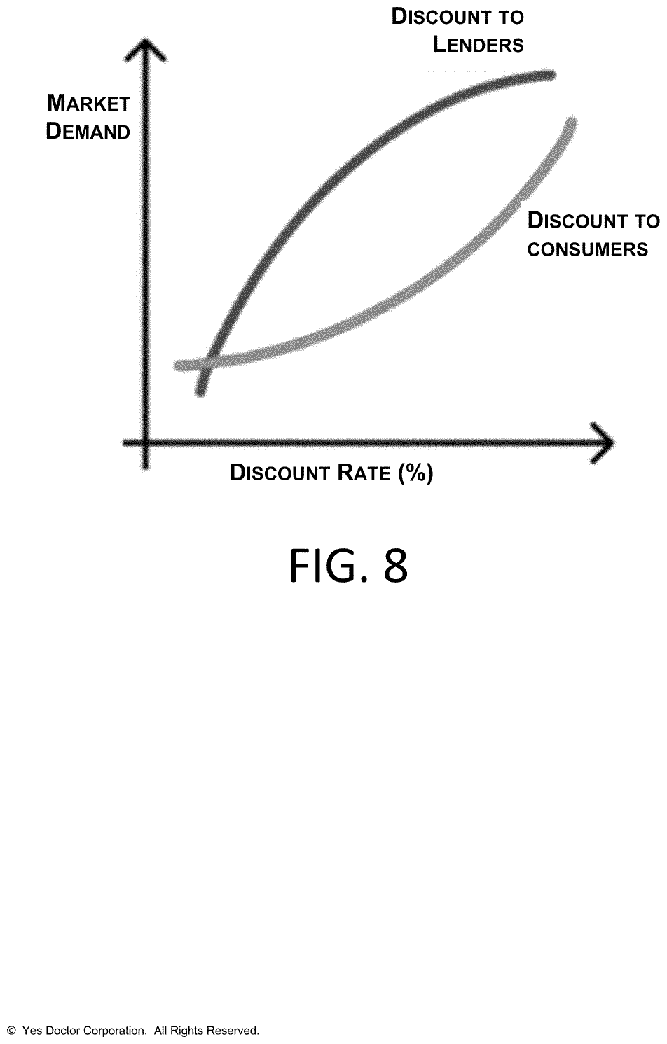

With reference to FIGS. 8 and 2B, the overall booking flow may be implemented on the platform by booking flow mechanism(s) 230. Case 1 may be implemented by the mechanism(s) regular booking flow for a known doctor 232; case 2 may be implemented by the mechanism(s) regular booking flow for an unknown doctor 234; case 3 may be implemented by the mechanism(s) capacity-optimized booking flow for a known doctor 236, and case 4 may be implemented by mechanism(s) capacity-optimized booking flow for an unknown doctor 238.

In some implementations, the platform 108 may regularly (e.g., daily) evaluate each clinic's/practitioner's schedule to determine if any patients in the system could fit into any available slots. This process may include running the booking flows 230 (FIG. 9A) for every clinic/practitioner and for every patient in the system. In this way, available slots may be filled if matches can be found.

Having matched a patient with an appropriate financial product (e.g., loan) and discount rate (provided by the clinic/practitioner to the lender), the patient may proceed with their procedure.

### DISCUSSION

In the disclosed methods, clinics offer a discount to lenders who will then finance the clinic service for the patients, e.g., with a 0% APR installment loan. It is understood that favorable financing options that spread the payments for a service over time and minimize the impact on the customers' cash flow will generate a higher demand for the service. In addition, a small increase in discount may enable a lender to approve a significantly larger population who will qualify for a financing product, increasing the population pool that the clinic can serve.

FIG. 8 shows the impact of the higher market demand that can be generated by offering the discount to the lender instead of directly to the customers in the market.

In a two-sided marketplace for a product or service, the demand for that product or service and the size of the potential customer pool both grow if the product is discounted. The demand grows as the discount grows.

By offering the discount to the lenders, the service price offered to the customers will be the original undiscounted price, and the clinic will be able to maintain the market perception of the original service price it offers.

The disclosed platform integrates and automates in part or in full the proposed discount and capacity optimization. The platform may also be a lender or integrate third-party lender services to offer financing.

Implementations or embodiments of the disclosed discount and capacity optimization may contain some or all of the following acts that may be integrated into or with the platform:
Agree with each participating practitioner (e.g., doctor) or clinic on a maximum discount rate, e.g., a percentage they are willing to offer to the lender(s).
Identify or calculate the possible financial products and a Minimum Required Discount Rate needed for a potential patient, e.g., as in FIG. 5.
Identify or calculate the probability of booking for a given unfilled capacity slot of a given clinic as a function of the discount rate the clinic offers, e.g., as in FIG. 6.
   The probability of booking may be estimated based on a machine learning or other algorithm or from past data of the clinics or may be learned over time.
   The probability of booking or filling a calendar spot is typically a monotonically non-decreasing function of the discount rate, e.g., as in FIG. 7.
Identify or calculate the optimum discount rate for the given calendar slot and given its probability of booking by optimizing the expected net profit of the clinic, e.g., as in FIG. 3A.
   The net profit of a clinic is a function of the discount rate that can be calculated from two functions: (i) the profit of the clinic conditioned or given the calendar slot is booked and (ii) the probability of booking the calendar slot. (i) is a decreasing function of the discount rate, and (ii) is a monotonic and non-decreasing function of the discount rate. Therefore, the expected net profit of a clinic for a given calendar spot as a function of the discount rate is expected to have a maximum, e.g., as shown in FIG. 4. The Optimum Discount Rate is the discount rate that results in the maxima.
Identify or calculate the Optimum Discount Rates for any number of open calendar slots (e.g., days) in the calendars of any number of clinics, e.g., as in FIG. 3B.
Identify or match patients and clinics where the patient's Minimum Required Discount Rates is less than or equal to the clinics' Optimum Discount Rates.

### Two-Sided Market Negotiation and Optimization

In a two-sided market, a service provider provides a service to a client. A two-sided marketplace serves a market by making a collection of service providers available to clients and facilitating a match and eventual transaction between a service provider and a client.

Exemplary embodiments may provide or include a system and method that may be a partially or fully automated workflow for negotiation and optimization of price or discount and or optimization of profit and or maximizing the probability of close of a service transaction in a two-sided marketplace. The workflow may include a financing-driven approach where the client is offered financing to pay for the services, e.g., through a 0% APR retail installment loan, and the service provider offers a discount to the lender that finances the service. Optimizing the discount rate from the service provider to the lender can maximize profit for the service provider and provide the client with the best or most affordable option to pay for the service.

The service provider may be a clinic or a doctor. The client may be a patient.

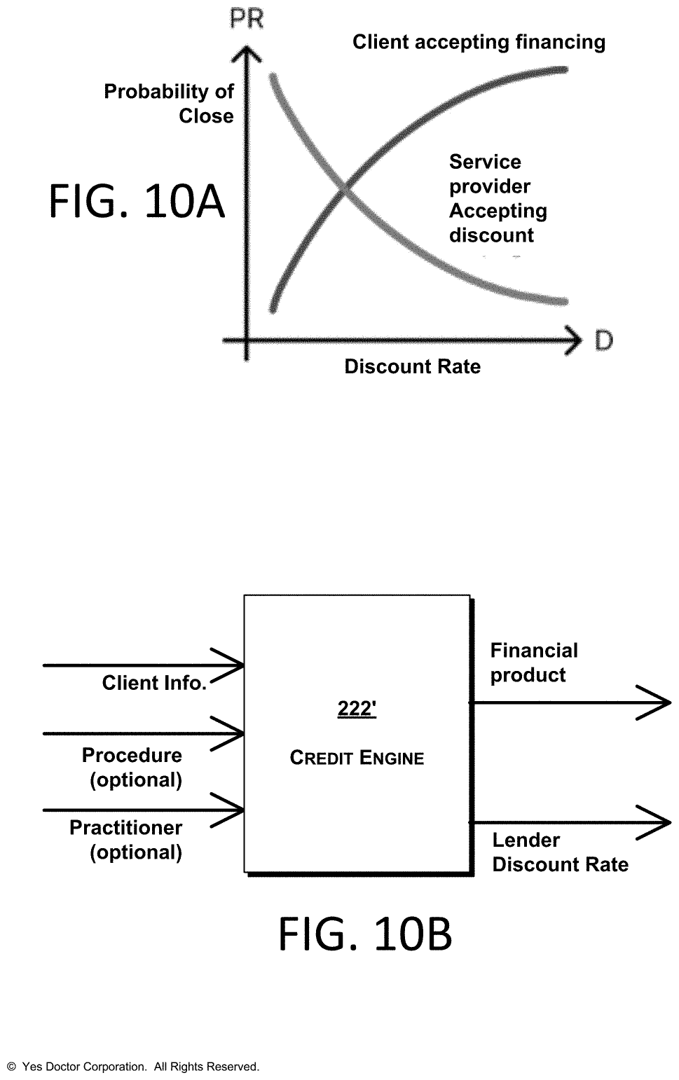

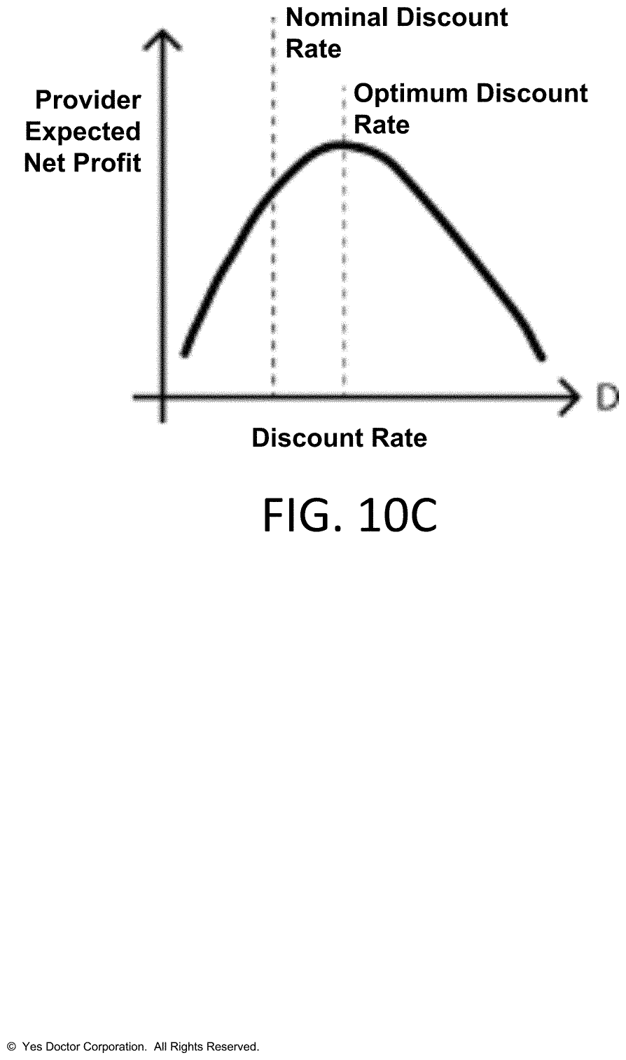

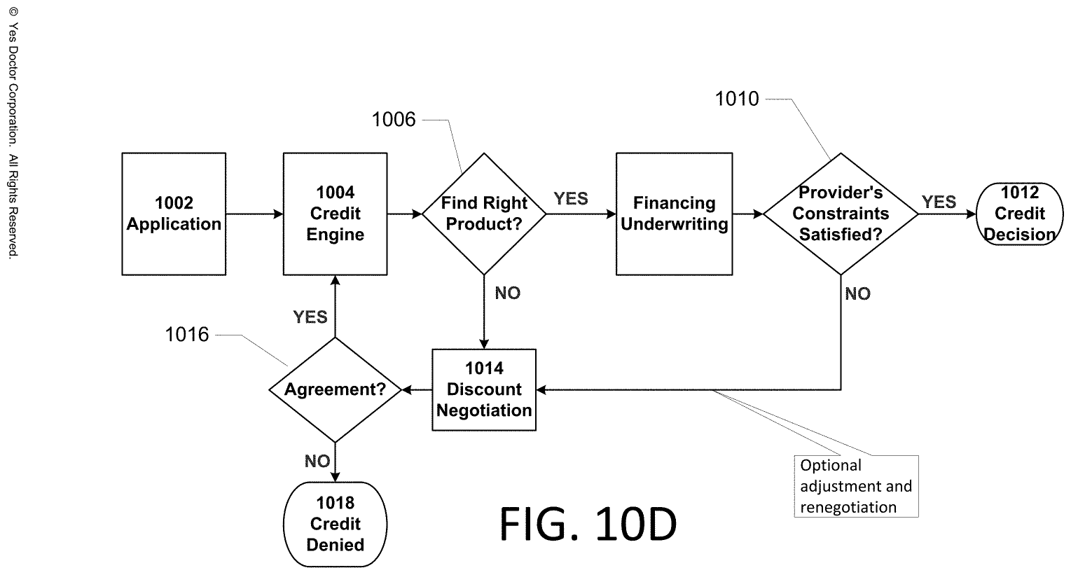

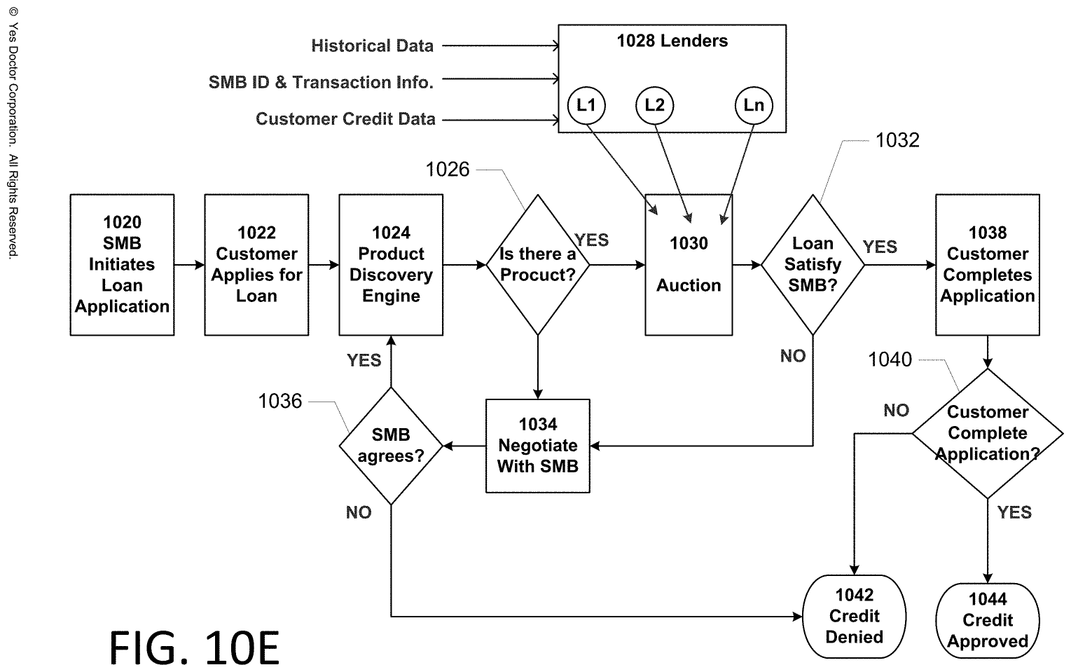

When the service provider offers a higher discount to the lender, the lender can offer better financing terms to the client, and the probability of accepting the financing by the client and closing the transaction increases. On the other hand, the probability of a service provider offering a discount decreases for higher discounts. This trade-off is shown in FIG. 10A.

Embodiments hereof provide a platform that integrates the system and method for automation of the workflow, negotiation, and optimization of the discount rate.

The discount rate optimization may contain some or all of the following steps that may be integrated into the platform:

Agree with each participating doctor or clinic on a nominal discount rate, e.g., in percentage that they are willing to offer to the lender(s)

Identify or calculate the possible financial product(s) and a Lender Discount Rate needed for a potential client, e.g., by a Credit Engine in the platform, as in FIG. 10B. Credit engine 222′ in FIG. 10B may be an instance of underwriting engine 222 in FIG. 5, discussed above.

Identify or calculate the probability of closing or accepting financing or discount by the service provider as a function of the discount rate the clinic offers, e.g., as in FIG. 10C.

The probability of closing may be estimated based on machine learning or other algorithm or from past clinics' data or may be learned over time.

Identify or calculate the probability of closing or accepting financing by a client as a function of the discount rate that the clinic offers

The probability of closing may be estimated based on machine learning or other algorithm or from client data, e.g., credit data.

Identify or calculate the optimum discount rate given the probability of close.

For example, the optimum discount rate may be found by optimizing the expected net profit of the clinic from a transaction for a calendar spot, e.g., as in FIG. 4.

The net profit of a clinic is a function of the discount rate that can be calculated from two functions: (i) the profit of the clinic conditioned or given the probability of closing by the client, (ii) the probability of closing by the client. (i) is a decreasing function of the discount rate, and (ii) is a monotonic and non-decreasing function of the discount rate, e.g., in FIG. 10A. Therefore, the expected net profit of a clinic as a function of the discount rate is expected to have a maximum, e.g., as shown in FIG. 4. The Optimum Discount Rate is the discount rate that results in the maxima.

If the optimum discount rate is higher than the nominal discount rate initially agreed with a service provider, there may be a discount rate negotiation for the given client or the given calendar slot. The discount negotiation may result in an updated agreement on the discount rate for the given client and the given calendar spot.

There may be financing underwriting to offer the financing to the client. The financing underwriting may include offering a single lender the agreed-upon optimum discount rate. Alternatively, the financing underwriting may include an auction for lenders, e.g., through bidding by multiple lenders who will participate in the auction to offer their pricing for the financing offered to the client.

There may be a final step to ensure the service provider constraints, e.g., discount rate, are met. For example, the best discount rate found in the auction by the winning lender is compared with the service provider's optimum discount rate. There may be an optional negotiation with the service provider if needed.

An example workflow is shown in FIG. 10D. An example workflow, including an auction step for financing, is shown in FIG. 10E.

As shown in the exemplary workflow in FIG. 10E, the SMB initiates a loan application (at 1020). The customer applies for a loan (at 1022). A product discovery engine is run (at 1024) to find a loan product that satisfies the SMB's requirements, regulations, etc. The product discovery engine may produce a first probability value (P1) that the borrower will accept the loan terms and a second probability value (P2) that the auction outcome will be within constraints agreed with the SMB.

If a product is found (at 1026) (e.g., if there is a product with P1 and P2 above certain thresholds), then the process continues with an auction (at 1030). If no product is found (at 1026), then there may be a negotiation with the SMB (at 1034), and if the SMB agrees (at 1036), then the product discovery engine is rerun (at 1024). If the SMB does not agree (at 1036), then credit is denied (at 1042).

Negotiation with the SMB (at 1034) may be to relax their constraint(s) to bring P1 and P2 above the required thresholds.

After the auction (at 1030), a check is made (at 1032) to determine whether the loan satisfies the SMB. If the loan does not satisfy the SMB (at 1032), then there may be a negotiation with the SMB (at 1034). If the loan does satisfy the SMB (at 1032), then the customer completes the application (at 1038), and if the customer's application is complete (at 1040), then the credit is approved (at 1044), otherwise, the credit is denied (at 1042).

### Integrated Patient Acquisition with Closed-Loop Global Optimization

Exemplary embodiments may provide or include a complete, integrated patient acquisition platform that includes marketing and financing. It also includes collecting feedback or data from patients and clinics and data from other sources, such as marketing channel analytics. The platform uses a global optimization method that minimizes the total patient acquisition cost. The total patient acquisition cost includes the cost of a patient lead, the conversion rate of the patient lead in the clinic, and the opportunity cost of a patient lead that does not convert. The cost of the patient lead includes the cost of acquiring a potential lead from a marketing channel, e.g., the cost per click for a Google AdWord or a Facebook ad, and the conversion rate of a patient leads to a patient who completes a treatment.

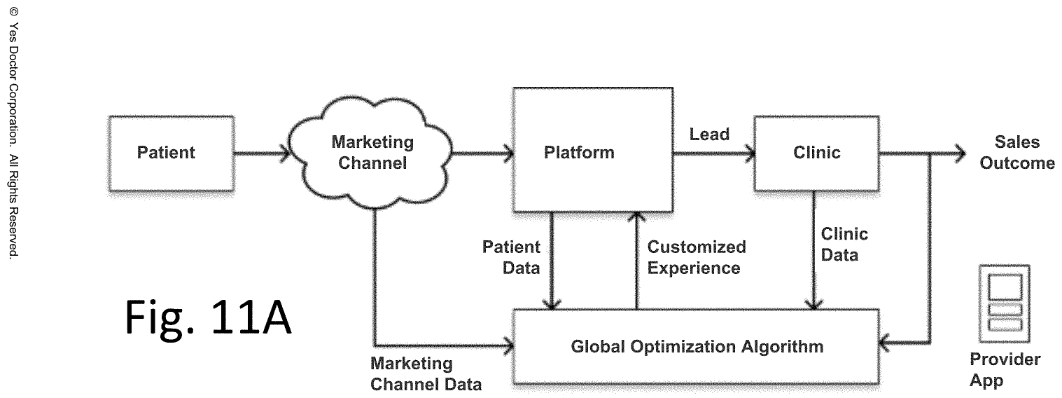

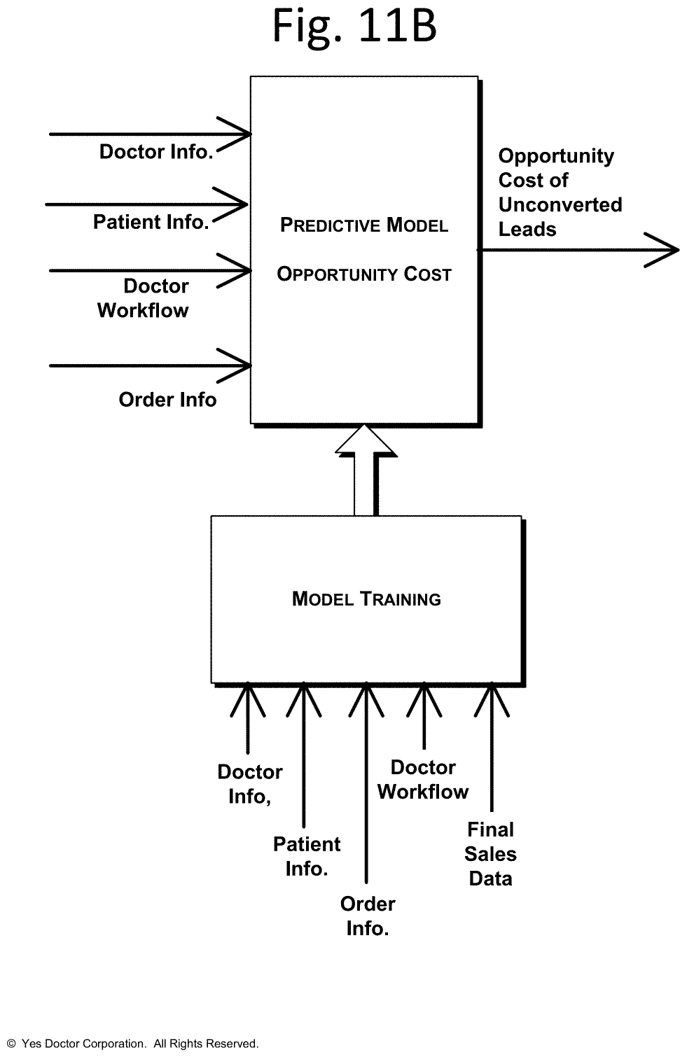

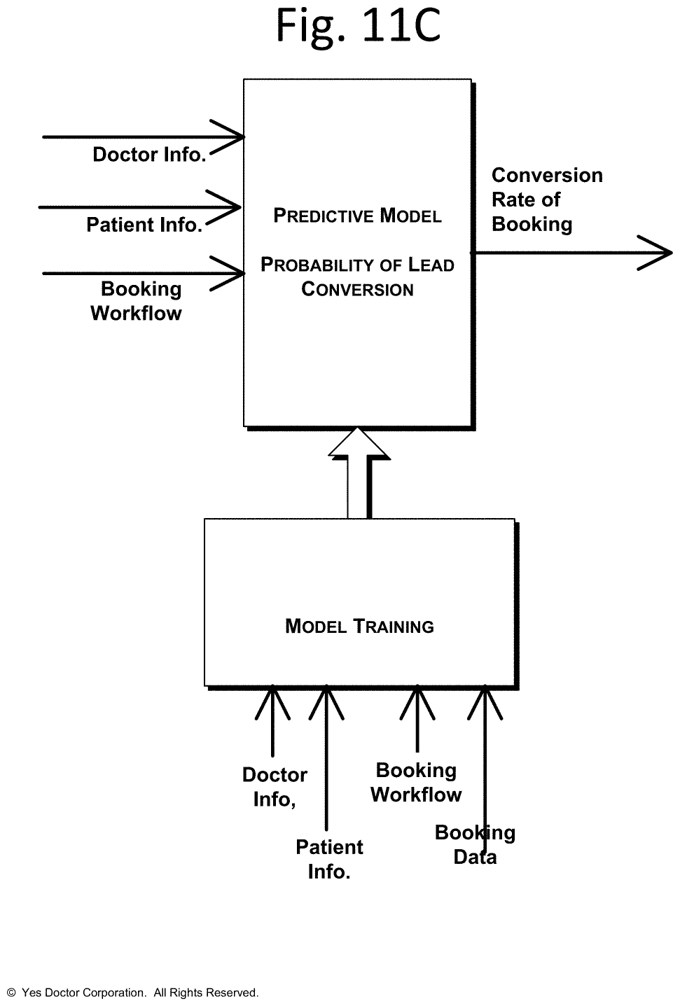

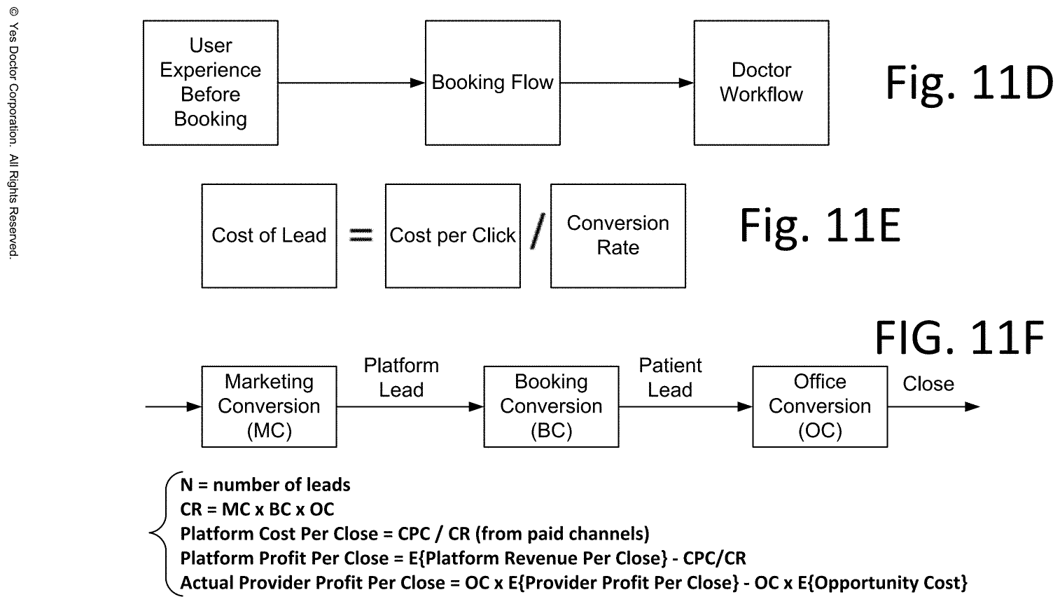

An example integrated patient acquisition platform with closed-loop global optimization is shown in FIG. 11A.

The platform may use machine learning, AI, and/or predictive modeling to estimate opportunity cost, lead conversion probability, conversion rate, or other parameters. Examples are shown in FIGS. 11B and 11C.

FIG. 11D shows various steps of the conversion. In each step, the cost of conversion for the platform and the information to which the platform has access varies from other steps. As such, the optimization algorithm and the cost functions may be different for each step. For example, before booking, optimization may focus on cost per click. In contrast, the booking flow optimization may focus on identifying leads with a low probability of conversion that could negatively impact the opportunity cost for the clinic.

FIG. 11E shows calculation for the cost of a lead according to exemplary embodiments hereof.

FIG. 11F shows various conversion stages and the impact of opportunity cost on the actual profit that clinics will generate.

The patient or user feedback or data may include (i) credit data, (ii) behavior in the platform such as length of time spent on any page, time and frequency of visits, pages visited, mouse movements, (iii) demographics and analytics data such as location, (iv) patient inputs and user entered data to the platform (v) patient selected procedure or treatment and other aspects of the patient order, (vi) or alternative data. The clinic feedback or data may include (i) feedback to indicate various states of a patient, including whether the patient lead has become a paying patient, (ii) clinic calendar and capacity, (iii) historical data of the clinic, (iv) other alternative data about the clinic.

The optimization method may utilize various modules, including (i) financing-driven marketing to reduce the Cost Per Click (CPC) for acquiring patient leads, (ii) a financing prequalification for patient leads, (iii) a patient lead questionnaire, (iv) a set of required or optional tasks for patient lead before visiting the clinic, (v) a mobile or web software application for potential patient clients, (vi) a mobile or web software application for clinic clients (Clinic App), (vii) a mechanism such as a UI in the Clinic App for clinics to provide feedback to the platform, (viii), a machine learning model that will learn from the user feedback or data including the behavior of the potential patients or the behavior of clinics or doctors throughout their engagement with the platform, (ix) other marketing or alternative data available.

The global optimization may also propose a customized experience on the platform for the patients and or for clinics to optimize conversion and patient acquisition costs.

The platform optimization method may require patients to fill out forms, fill questionnaires, or provide information for soft or hard credit pull and then use the information to pre-qualify patients financially or to check if patients satisfy other minimum requirements, such as medical requirements defined by doctors or by clinics, for a procedure.

The optimization method may consider a variable opportunity cost for different clinics based on patient or clinic data, e.g., patient procedure, clinic calendar, and clinic capacity.

### Financing-Driven Marketing and Financing Prequalification for Patient Acquisition

Exemplary embodiments may provide or include a new patient acquisition system and workflow that may include any of (i) a marketing step in the patient acquisition workflow where the financing options are advertised or offered to the entire pool of potential patients, (ii) an integrated financing prequalification step in the patient acquisition workflow during which the potential patients are pre-qualified to receive available financing options. The prequalification step may be added at the top or close to the top of the patient acquisition funnel and before the potential patient makes an appointment with or visits a provider or a clinic, (iii) the delivery of the potential patient's prequalification information to the clinic, (iv) indirect loans, e.g., retail installment loan, to patients to cover all or part of the cost of service.

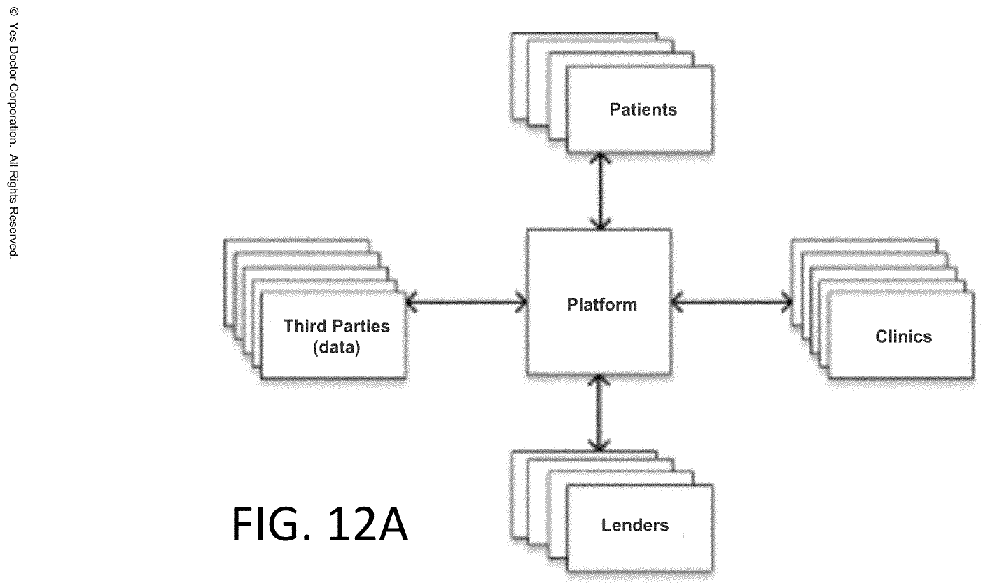

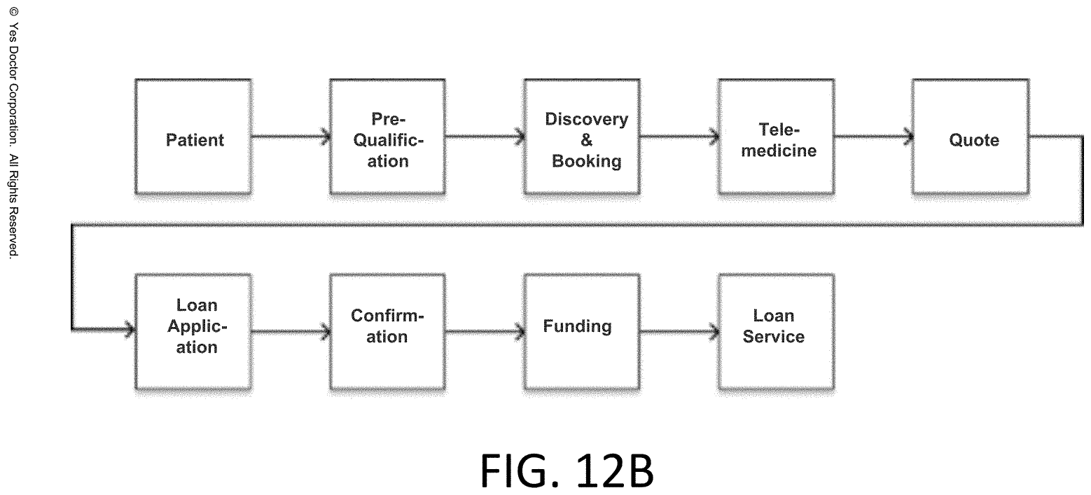

The new financing-driven marketing platform engages and serves all parties and information in the ecosystem to complete a patient journey for treatment. The parties include patients, clinics, lenders, and third parties, as shown in FIG. 12A. The patient workflow for financing-driven marketing may include all or some steps shown in FIG. 12B.

The patient acquisition workflow may include any of several modules, components, or processes, including:
Marketing, e.g., advertisement or digital marketing on various platforms such as social media or search engines to create potential patient leads
Client or patient user interface (UI), e.g., a web application or a mobile application
Prequalification, e.g., by entering name and address or other required personal information for a hard credit pull or a soft credit pull on the use of such credit information as well as other relevant information to pre-qualify the potential patients for the use of financing options for financing products.
Discovery, e.g., exploring through the UI digital content such as text, images, videos, calendar data, reviews, scores, questions and answers, and lists. Such content may include information about doctors, clinics, procedures, treatments, other patient journeys or stories, a community or social network of existing patients, or other content that helps potential patients learn, decide, or complete a procedure.
Booking, e.g., making an appointment through UI for virtual or in-person consultation with a clinic, clinic staff, or a doctor.
Consultation, e.g., a virtual session for a meeting between a potential patient and a doctor or a clinic staff to exchange information about a potential treatment for the potential patient.
Quote, e.g., providing the cost of the potential treatment to the potential patient, which may be broken down into multiple line items and may include the financing option and detail of the financing terms
Payment, e.g., processing online or mobile payment by credit card, so the patient can pay for all or portions of the amount due for an appointment, booking fee, or completing a procedure.
Financing Application, e.g., the application workflow so that a potential patient may apply and receive financing to complete the given treatment with the given doctor or clinic staff at the given clinic
Treatment workflow management, e.g., the process and tools including UI and software applications so that the clinic staff or doctor can manage the treatment workflow, including (i) engaging potential patients and providing information, e.g., the treatment quote or required information, e.g., required medical tests or medical information from the patient, (ii) review the information provided by the potential patient, (iii) access appointments and start and conduct virtual consultation with potential patients, (iv) record information or provide input or feedback to patient acquisition platform about the various steps of the treatment, e.g., pre-operation, surgery, or post-operation appointment dates.
Funding and Loan Servicing, e.g., fund the financing amount from the lender to the clinic or doctor and collect the required payments related to financing from the patient.
PMS Integration, e.g., integration with third-party practice management systems (PMS) to exchange information automatically between potential patients and clinics or doctors.

The integrated financing may be in the form of direct lending or indirect lending. Indirect lending may be in the form of a retail installment loan to the patient. The platform integrates and automates all or parts of financing, e.g., underwriting, funding, servicing, or integration with third-party lenders.

### Automating and Optimizing the Full-Cycle Workflow of Engaging, Acquiring, and Serving Patients

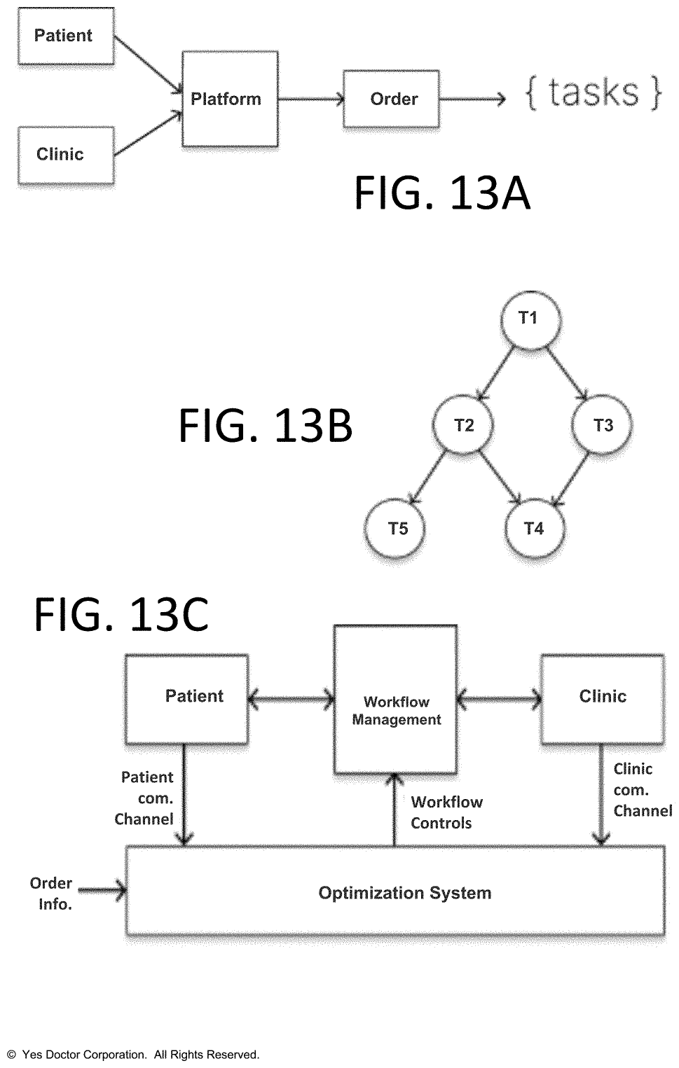

Exemplary embodiments may provide or include an integrated platform for automated and optimized workflow management for the complete cycle of engagement between a patient and a clinic or doctor. When the potential patient and the clinic and or doctor are engaged through the platform, the interaction and all information and processes related to the engagement and interactions and other related data are included in an Order entity or Order object in the platform. For example, the Order may include information about the patient, the clinic, the doctor, the appointments, the financial statement(s) and payment(s), and the loan(s), all associated with the specific Order. To complete an Order, which includes providing the services related to the treatment and payments for treatment, one or multiple workflows may need to be executed. A workflow includes a collection or list of one or several tasks that may need to be done sequentially or in parallel for the execution of the workflow. Tasks may be assigned to one or several entities, such as the patient, the doctor, the clinic staff, or the platform admin See, e.g., FIG. 13A.

Tasks may have one or several attributes, such as description, assignee, due date, or status. The task status may be one of several values, such as new, active, complete, or obsolete. The task status may have the following definitions:
New: A new task is a task that is part of the workflow but is not yet started or activated.
Active: An active task is a task that has started or has some pending actions by its assignee or is pending a trigger event, e.g., a time event trigger. When a task becomes active, the workflow management system, in tandem with other modules in the platform, may notify assignees, create or activate some triggers, or do some other actions, and then monitors the actions or trigger events that are needed for a task to be done
Completed: a completed task is a task for which all its required actions or events for its completion are done
Expired: an expired task is a task in a workflow that was new or active but no longer needed to be completed.
   Task status={New, Active, Completed, Expired}

The workflow may be defined such that tasks in the workflow may have a required sequence or dependency on one or multiple other tasks, e.g., the workflow may require that the patient task to join a virtual consultation session cannot be completed or even become active before (i) the patient task to fill a medical questionnaire is completed and (ii) the doctor task to confirm the review of such questionnaire is also completed.

The status of a task may be updated when a trigger event occurs. The trigger event may be an instance of time. For example, the task of sending a reminder to a patient who has an appointment on Nov. 20, 2023, may be triggered 24 hours before the appointment. The trigger event may be a change of status of another task. For instance, the task for a patient to review and sign a quote sent by the clinic may become active when the status of the task for the clinic to send the quote is changed to completed.

The workflow management system is part of the platform that can monitor the state of the Order(s) in the platform, can manage the status of the tasks in each of the workflows of each of the Orders, and or automates the execution of all or parts of the workflows. This workflow management and automation reduce significant amounts of complexity and overhead from clinics and create a frictionless and convenient experience for the patients and clinics.

In a workflow, the list, the sequence, interdependence, or the attribute of tasks may be predetermined, e.g., for each clinic, and may be programmed in the platform. An example workflow A, WF_A, is shown below. It consists of 5 tasks, T1, T2, T3, T4, and T5. The WF_A has a defined sequence. T2 and T3 can occur in parallel, occurring after T1 is completed. T4 can occur if both T2 and T3 are completed. See, e.g., FIG. 13B.

The workflow management system may automate the execution of the workflow.

For example, in one way, in Forward-Looking Workflow Automation (FLWA), all workflow tasks are in new states, and some may become active, e.g., if they are first in a sequence. When the system updates the status of a task, e.g., when an active task is completed, the system then changes the status of all the tasks that are next in sequence to or dependent on the completed task to active. However, the system must also check if each of the tasks that are next in sequence to the completed tasks is dependent on other tasks that are prior in sequence and, if so, if such prior tasks are also completed. For example, in WF_A above, After T2 is completed, the system can make T4 and T5 active. However, before making T4 active, the system should check if T3 is completed or not. If T3 is still active, T4 cannot become active.

In another way, Backward-Looking Workflow Automation (BLWA), all workflow tasks are in new states, and some may become active, e.g., if they are first in a sequence. Then, as a result of a trigger event, e.g., when an active task is completed, the system may check all or a subset of all tasks to see if the prerequisites for change of status, e.g., to active or complete of any of such tasks are met. If yes, the system updates the status of those tasks.

The workflow does not have to be predetermined or fixed and may be optimized over time. The optimization of the workflow may help to create a more convenient experience for the patient, to maximize the probability of conversion at various steps of the patient acquisition funnel, to enable adoption to a varying workflow in a clinic, minimize the workflow inefficiencies as they are better understood over-time, or to optimize for a combination of these and or other factors. See, e.g., FIG. 13C.

The optimization method may include a learning system that observes various data, e.g., data entered by the patient, the behavior of the patient or clinic when engaged with the platform, communication data between the patient and platform or clinic and platform, or data entered by the clinic.

Exemplary Implementation—Computing

The applications, services, mechanisms, operations, and acts shown and described above are implemented, at least in part, by software running on one or more computers or computer systems.

Programs that implement such methods (as well as other types of data) may be stored and transmitted using various media (e.g., computer-readable media) in several ways. Hard-wired circuitry or custom hardware may be used in place of, or in combination with, some or all of the software instructions that can implement the processes of various embodiments. Thus, various combinations of hardware and software may be used instead of software only.

Upon reading this description, one of ordinary skill in the art will readily appreciate and understand that the various processes described herein may be implemented by, e.g., appropriately programmed general-purpose computers, special-purpose computers, and computing devices. One or more such computers or computing devices may be referred to as a computer system.

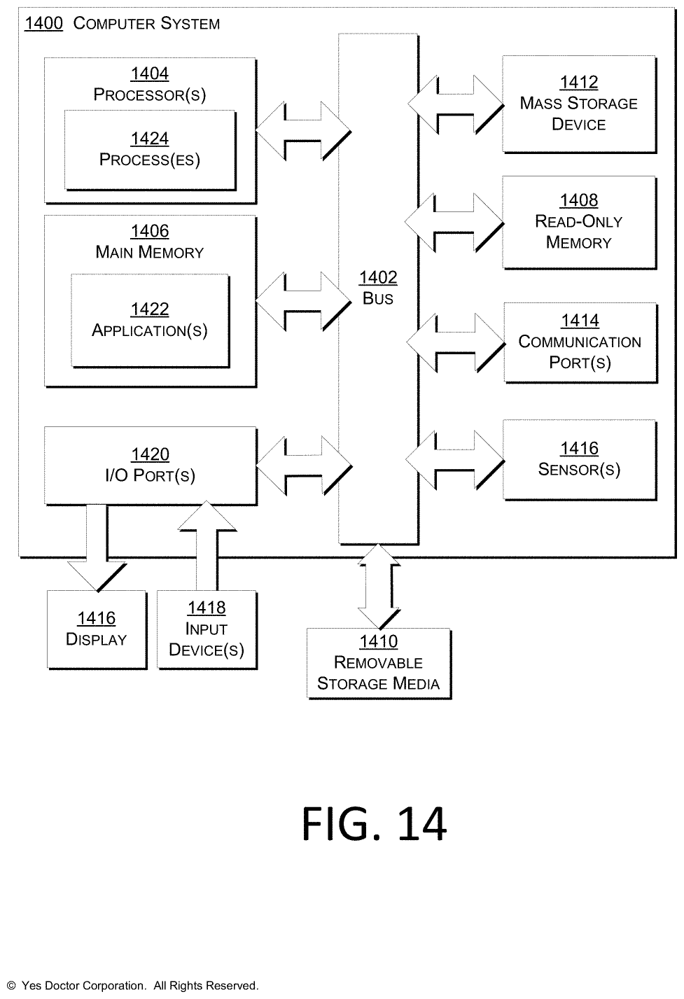

FIG. 14 is a schematic diagram of a computer system 1400 upon which embodiments of the present disclosure may be implemented and carried out.

According to the present example, the computer system 1400 may include a bus 1402 (i.e., interconnect), one or more processors 1404, one or more communications ports 1414, location device(s) 1415, a main memory 1406, optional removable storage media 1410, a read-only memory 1408, and a mass storage 1412. Communication port(s) 1414 may be connected to one or more networks (e.g., computer networks, cellular networks, etc.) by way of which the computer system 1400 may receive and/or transmit data. The location device(s) 1415 may include GPS devices and the like that can be used to determine the device's location.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input and output devices that are appropriate to perform the process.

Processor(s) 1404 can be (or include) any known processor, such as but not limited to, an Intel® Itanium® or Itanium 2® processor(s), AMD® Opteron® or Athlon MP® processor(s), or Motorola® lines of processors, and the like. Communications port(s) 1414 can be any RS-232 port for use with a modem-based dial-up connection, a 10/100 Ethernet port, a Gigabit port using copper or fiber, or a USB port, and the like. Communications port(s) 1414 may be chosen depending on a network such as a Local Area Network (LAN), a Wide Area Network (WAN), a Content Delivery Network (CDN), or any network to which the computer system 1400 connects. The computer system 1400 may be in communication with peripheral devices (e.g., display screen 1416, input device(s) 1418) via Input/Output (I/O) port 1420. Some or all peripheral devices may be integrated into the computer system 1400, and the input device(s) 1418 may be integrated into the display screen 1416 (e.g., in the case of a touch screen).

Main memory 1406 can be Random Access Memory (RAM) or any other dynamic storage device(s) commonly known in the art. Read-only memory 1408 can be any static storage device(s), such as Programmable Read-Only Memory (PROM) chips for storing static information, such as instructions for the processor(s) 1404. Mass storage 1412 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 1402 communicatively couples processor(s) 1404 with the other memory, storage, and communications blocks. Bus 1402 can be a PCI/PCI-X, SCSI, a Universal Serial Bus (USB) based system bus (or other) depending on the storage devices used, and the like. Removable storage media 1410 can be any kind of external hard-drive, Compact Disc—Read-Only Memory (CD-ROM), Compact Disc—ReWritable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) that may be read by a computer, a processor or a like device. Such a medium may take many forms, including but not limited to non-volatile media, volatile media, and transmission media. Non-volatile media include, for example, optical or magnetic disks and other persistent memory. Volatile media include dynamic random-access memory, which typically constitutes the computer's main memory. Transmission media include coaxial cables, copper wire, and fiber optics, including the wires that comprise a system bus coupled to the processor. Transmission media may include or convey acoustic waves, light waves, and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product. The program may be transferred from a remote computer to a requesting computer by way of data signals embodied in a carrier wave or other propagation medium via a communication link (e.g., modem or network connection).

Various forms of computer-readable media may be involved in carrying data (e.g., sequences of instructions) to a processor. For example, data may be (i) delivered from RAM to a processor; (ii) carried over a wireless transmission medium; (iii) formatted and/or transmitted according to numerous formats, standards, or protocols; and/or (iv) encrypted in any of a variety of ways well known in the art.

A computer-readable medium can store (in any appropriate format) those appropriate program elements to perform the methods.

As shown, main memory 1406 is encoded with application(s) 1422 that support(s) the functionality as discussed herein (an application 1422 may be an application that provides some or all of the functionality of one or more of the mechanisms described herein). Application(s) 1422 (and/or other resources as described herein) can be embodied as software code such as data and/or logic instructions (e.g., code stored in the memory or on another computer-readable medium such as a disk) that supports processing functionality according to different embodiments described herein.

For example, as shown in FIG. 2A, application(s) 1422 may include underwriting mechanism(s) 222, model training mechanism(s) 224, expected net profit optimizer mechanism(s) 226, booking probability estimation model mechanism(s) 228, and booking flow mechanism(s) 230.

During the operation of one embodiment, processor(s) 1404 accesses main memory 1406, e.g., via bus 1402, to launch, run, execute, interpret, or otherwise perform the logic instructions of the application(s) 1422. Execution of application(s) 1422 produces processing functionality of the service(s) or mechanism(s) related to the application(s). In other words, the process(es) 1424 represents one or more portions of the application(s) 1422 performing within or upon the processor(s) 1404 in the computer system 1400.

For example, process(es) 1424 may include process(es) corresponding to one or more of the application(s) 1422.

It should be noted that in addition to the process(es) 1424 that carries(carry) out operations as discussed herein, other embodiments herein include application 1422 (i.e., the un-executed or non-performing logic instructions and/or data). The application 1422 may be stored on a computer-readable medium (e.g., a repository) such as a disk or optical medium. According to other embodiments, the application 1422 can also be stored in a memory type system such as in firmware, read-only memory (ROM), or, as in this example, as executable code within the main memory 1406 (e.g., within Random Access Memory or RAM). For example, application 1422 may also be stored in removable media 1410, read-only memory 1408, and/or mass storage device 1412.

Those skilled in the art will understand that the computer system 1400 can include other processes and/or software and hardware components, such as an operating system that controls the allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, including hardware, software, firmware, or any combination thereof.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

### CONCLUSION

As used herein, including in the claims, the phrase “at least some” means “one or more” and includes the case of only one. Thus, e.g., the phrase “at least some ABCs” means “one or more ABCs” and includes the case of only one ABC.

As used herein, including in the claims, the phrase “based on” means “based in part on” or “based, at least in part, on” and is not exclusive. Thus, e.g., the phrase “based on factor X” means “based in part on factor X” or “based, at least in part, on factor X.” Unless specifically stated by the use of the word “only,” the phrase “based on X” does not mean “based only on X.”

As used herein, including in the claims, the phrase “using” means “using at least” and is not exclusive. Thus, e.g., the phrase “using X” means “using at least X.” Unless specifically stated by the use of the word “only,” the phrase “using X” does not mean “using only X.”

In general, as used herein, including in the claims, unless the word “only” is specifically used in a phrase, it should not be read into that phrase.

As used herein, including in the claims, a list may include only one item. Unless otherwise stated, a list of multiple items need not be ordered in any particular manner Unless specifically stated otherwise, a list may include duplicate items. For example, as used herein, the phrase “a list of XYZs” may include one or more “XYZs.”

It should be appreciated that the words “first” and “second” in the description and claims are used to distinguish or identify and not to show a serial or numerical limitation. Similarly, words such as “particular,” “specific,” “certain,” and “given,” if used, are to distinguish or identify within a claim and are not intended to be otherwise limiting. Furthermore, letter labels (e.g., “(A),” “(B),” “(C),” and so on, or “(a),” “(b),” and so on) and/or numbers (e.g., “(i),” “(ii),” and so on) if in the claims, are used to assist in readability, and are not intended to be otherwise limiting or to impose any serial or numerical limitations or orderings.

Unless specifically shown and stated, no ordering is implied by any labeled boxes in any flow diagrams. When disconnected boxes are shown in a diagram, the activities associated with those boxes may be performed in any order, including fully or partially in parallel.

Thus are described methods, devices, and systems supporting the underwriting and financing of elective health procedures for clinical capacity optimization. While the invention has been described in connection with what is presently considered to be the most practical and preferred embodiments, it is to be understood that the invention is not to be limited to the disclosed embodiment but, on the contrary, is intended to cover various modifications and equivalent arrangements included within the spirit and scope of the appended claims.

We claim:

1. A method, in a system in which one or more practitioners associated with a clinic offer services or procedures, each service or procedure requiring one or more time slots, each service or procedure having a rate associated therewith,
the method comprising:
(A) obtaining a calendar identifying available time slots for at least some of said one or more practitioners;
(B) obtaining a list of one or more potential patients, each potential patient desiring at least one service or procedure;
(C) determining, for at least some of said available time slots in said calendar, a corresponding optimum discount rate; and
(D) determining, for each of said at least some available time slots in said calendar, an effective net profit as a function of said corresponding optimum discount rate, wherein the optimum discount rate for a calendar slot is the discount rate that maximizes an expected net profit of the clinic;
(E) for a particular potential patient on said list of one or more potential patients, determining possible financial products and/or a minimum required discount rate; and then
(F) offering said particular potential patient one of said available slots at said optimum discount rate determined in (C).

2. The method of claim 1, wherein each practitioner has a maximum discount rate, and wherein the optimum discount rate for each available time slot for a particular practitioner does not exceed that particular practitioner's maximum discount rate.
